(19) United States
(12) Patent Application Publication
(10) Pub. No.: US 2019/0383730 A1
(43) Pub. Date: Dec. 19, 2019

(54) MULTISPECTRAL IMAGE ANALYSIS SYSTEM

(71) Applicant: TAIWAN HIPOINT CORPORATION, Kaohsiung City (TW)

(72) Inventor: Chin-Pin CHANG CHIEN, Kaohsiung City (TW)

(21) Appl. No.: 16/151,048

(22) Filed: Oct. 3, 2018

(30) Foreign Application Priority Data
Jun. 16, 2018 (TW) 107208156

(51) Int. Cl. G01N 21/25; G01N 33/00

(52) U.S. Cl. CPC G01N 21/255; G01N 2021/1765; G01N 33/0098

(57) ABSTRACT

A multispectral image analysis system includes a carrier, a power controller, a stroboscope, a spectrum transducer, a multispectral light control system, a whiteboard calibrator, an environmental control system, a man-machine interface controller, at least an optical photography system and an image capturing and analyzing system. By the present invention, a plurality of target samples can be cultivated simultaneously under the control of a stable environmental condition. In addition, using the multispectral light of the present invention to observe all kinds of physiologic and pathologic features of the target samples, the differences in the phenotype and spectrum for a plurality of the target samples are accessed for comparison, which can be used as a reference for analysis and determination.

### BACKGROUND OF THE INVENTION

### a) Field of the Invention

The present invention relates to a cultivation and automatic monitoring analysis system for a massive amount of biological samples. The system is characterized in having an environmental control system to fix the locations of the biological samples for cultivation in a long and periodical time, and having a multispectral light control system to provide a specific multispectral light source for photographing the phenotypes of the biological samples.

### b) Description of the Prior Art

Due to the advancement and well development of technology, telemetry has been widely used in a daily life. For example, a weatherman will use the cloud images to explain the weather in the future, a surveyor will use the aerial photographs to make the maps, an environmentalist will collect the environmental information using the telemetry and an agriculturist will identify the telemetric images to monitor the growth of the field crops. As the telemetry is able to collect the data in a large range in the shortest time, it has already become the indispensable tool to understand the earth. In general, the telemetry is a kind of technology for collecting the data of a target using the sensors without contacting the target, and is divided into the active telemetry (Lidar or microwave telemetry) and the passive telemetry (optical telemetry) depending on the sensors. To collect the data in a large range, a carrier such as airplane or satellite will carry the sensors in the air to detect the ground, ocean or atmosphere, and the data will be recorded for analysis. Furthermore, hyperspectral imaging is a kind of optical telemetry, using the differences in the reflection value of the absorption of sunlight at various wavebands for all materials to develop a unique spectral reflection curve for all materials. The spectral reflection curve can be used to analyze and identify a specific material, solving the problem that a pure picture can cause a misjudgment at a same color.

In terms of the development of the sensor technology, the spectral resolution of the telemetry images has been improved from a few or several dozens of the wavebands for the old multispectral imaging, to several hundreds of wavebands for the existing hyperspectral imaging. Due to the improvement in the spectral resolution, the spectral curves of the collected data of materials will be even closer to the real spectral curves of the materials, which facilitates the detection and classification of materials. The applications of the hyperspectral imaging are divided primarily into three kinds, including the target identification, the description of background features and the anomaly detection. The target identification includes the target detection, target identification and target classification, and is used in the change detection and the monitoring of material status. The description of background features aims at the coverings on the earth surface, the hydrology and the atmosphere, and is applied to the detection of coast lines and the buildup of the topographic textures in shallow water. Finally, the anomaly detection is to find out a target of which the spectrum is explicitly different from the spectrum of background in an unknown area. In using the hyperspectral imaging to perform the analysis and monitoring, the first problem is that as the ground resolution is not sufficient, many signal sources will be embedded in a same pixel. Therefore, the conventional image processing technology based upon the space is no longer useful, and instead, the subpixel detection method based upon the spectrum is used to deal with this kind of problem, followed by performing the analysis and monitoring to the target using the space or spectrum. As there can be more than two materials in a pixel, the pixel will be no longer a pure pixel, and the conventional pure pixel classification method will not be able to solve this kind of problem effectively. Thus, each pixel should be taken as a mixed pixel, and is then processed by the linear spectral mixture model. The linear spectral mixture model is a technology which is widely applied to the telemetry imaging to detect and quantify the individual material, wherein the pixel spectrum model is built up assuming that the content of each material in every pixel is a linear distribution. After building up the spectrum model, a proper method is used to solve this linear equation, and then the material identification, detection, classification and quantification can be accomplished.

The hyperspectral imaging has been applied more and more widely and the primary applications nowadays are the terrain identification, agriculture cultivation, hydrology detection, ecological environment monitoring and military target detection. Therefore, almost all of the telemetry specialists in the world are involved in the development and research of this knowledgeable imaging. However, the bandwidth of hyper-spectrum is smaller, which is about 1-6 nm, and the wavebands are plenty. Therefore, the price is very expensive, and the data amount of the spectral images collected is huge that a supercomputer is required for analysis. In addition, the hyperspectral imaging is difficult in analyzing the images in a specific area in a small range and can only be applied to the agricultural image identification in a large area. Accordingly, the development of a low-priced hyperspectral imaging is getting attention significantly. Although its waveband is larger, about 60 nm, the hyperspectral imaging is sufficient for the phenotype analysis in a small area.

In the prior arts regarding the research on the spectral analysis to a tested object, Taiwanese Patent Publication Nos. 1405957, 1522607, 1592650 and 200946896 have disclosed a new spectral test method and structure, a mobile device for receiving the data of detection device, and a plasma generator for generating a spectrum to analyze the elements of tested object. These prior arts include an automatic image analysis technology of the biological growth media. In addition, in terms of the research on the image analysis to multi-sample organisms, a US Patent Publication No. US 20180004872 A1 has disclosed a method of identifying and classifying social complex behaviors among a group of model organisms, comprising implanting an RFID (Radio Frequency Identification) transponder in each model organism in the group of model organisms, capturing a sequence of images of each model organism over a period of time, and calculating the features of the model organism.

There is also a research on the image analysis to multi-sample organisms under a controlled environment, as disclosed in a US Patent Publication No. US 20120186154 A1, which is an automation useful in cultivating or phenotyping a plurality of plants in a controlled environment. The automation includes a plurality of movable cultivation substrate holders, and the holders can consecutively take every possible position on the substrate to reduce significantly the change in the cultivation environment or phenotype. However, in addition to that the cultivation substrate is affected by the environment, the organisms can be even more explicitly affected and changed by pests, and these changes cannot be observed only by the pure images. Accordingly, in terms of shortening the time of research on physiology and pathology, the spectral analysis has become an important topic in the development of biological technology.

### SUMMARY OF THE INVENTION

The present inventor has been working in the business of cultivation environmental control system and organism phenotype analysis for many years, and thus, knows very well that in the study of organism physiology and pathology, the quantity of samples is large, the time of reproduction test is long, and there is still some insufficiency in the wavebands of spectrum for the phenotype analysis requiring understanding. Accordingly, the present inventor is now endeavoring on the development of a multispectral image analysis system, and the present invention discloses a multispectral image analysis system, comprising a carrier, a power controller, a stroboscope, a spectrum transducer, a multispectral light control system, a whiteboard calibrator, an environmental control system, a man-machine interface controller, at least an optical photography system, and an image capturing and analyzing system. The carrier provides a holding space for the placement and cultivation of target samples, and is a containment which can be opened and closed by a cover, accommodating part or all of the power controller, stroboscope, spectrum transducer, multispectral light control system, whiteboard calibrator, environmental control system, man-machine interface controller and optical photography system. The power controller is connected with the stroboscope, the spectrum transducer, the multispectral light control system, the whiteboard calibrator, the environmental control system, the man-machine interface controller and the optical photography system to provide the power for operation. The stroboscope provides a flashlight to irradiate on the target samples, and the reflection light from the target samples aids the optical photography system to shoot images. The spectrum transducer is disposed in the irradiation range of the stroboscope and the multispectral light control system to monitor the wavelength and the illuminance, followed by sending back these data to the man-machine interface controller. The multispectral light control system is disposed above or beside the target samples, providing a multispectral light with 16-20 kinds of wavebands in an interval of 25-45 nm and with a power switch being controlled or a light intensity being adjusted by the man-machine interface controller. The whiteboard calibrator is disposed in the shooting range of lens of the optical photography system, measuring the coordinates of the target samples and sending back the data to the man-machine interface controller to calibrate the gray level and the focus in height, so as to provide the optical photography system with the quality of images to be shot. A part of the environmental control system is disposed inside and outside the carrier to provide heat exchange between the inner space and the outer space of the carrier, provide the inner space of the carrier with a stable environment, monitor the environment in the inner space of the carrier, and then send back the monitoring data to the man-machine interface controller. The man-machine interface controller links bi-directionally with the power controller, the stroboscope, the spectrum transducer, the multispectral light control system, the whiteboard calibrator, the environmental control system and the optical photography system to control the operation of all parts and receive the real monitoring data of all parts. The optical photography system is disposed above or beside the target samples to shoot the target samples and send back the images to the man-machine interface controller. The image capturing and analyzing system links bi-directionally with the man-machine interface controller to receive the real monitoring data of all parts from the man-machine interface controller for analysis, and then send back the data after analysis to the man-machine interface controller for the compensation or operation of the environmental control parameters. The holding space in the carrier provides for the placement and cultivation of a plurality of target samples for comparison and analysis. The wavelength of the spectrum of the multispectral light control system is in the range of 330-1100 nm, and the multispectral light control system can adjust a switch of a single light source and an intensity of the single light source. The environmental control system includes a temperature controller and a humidity controller to control the environmental condition such as temperature, humidity or salinity. The man-machine interface controller includes a display unit, a touch control unit and a press-key unit disposed on the outer surface of the carrier, allowing a user to monitor and input the control condition. The optical photography system is provided with a function of shooting 2-D (2-Dimensional) pictures, films, time-lapse photography, 3-D (3-Dimensional) pictures and images for the user to determine. Furthermore, the optical photography system includes a mechanical arm or a slide rail to move the lens of the optical photography system to shoot the target samples, wherein the mechanical arm or the slide rail can move at least front and back, left and right, or up and down, and provide for setting up and moving the lens and the stroboscope. The features of plants that are analyzed by the image capturing and analyzing system include the features of spectrum, the growth rate, the pathological analysis, the observation of cultivation phenotype, the diffusion rate, the diffusion and covering area and the proportion and distribution of the phenotype of a plurality of samples, with the images, coordinates and spectrum features collected providing multi-dimensional data for further data analysis. By the present invention, the plurality of target samples can be cultivated simultaneously in a long time under the control of a stable environmental condition. In addition, using the multispectral light of the present invention to observe all kinds of physiologic and pathologic features of the target samples, the differences in the phenotype and spectrum of the plurality of target samples can be accessed for comparison, which can be used as a reference for the determination of the timing of medication. The environmental control structure and the multispectral features are different from those in the prior arts, and therefore, the present invention is indeed provided with novelty, advancement and practicality.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a structural diagram of a multispectral image analysis system, according to the present invention.

FIG. 2 shows a schematic view of the structure of the multispectral image analysis system, according to the present invention.

FIG. 3 shows a first diagram of time phenotype analysis in the structure diagram of the multispectral image analysis system, according to the present invention.

FIG. 4 shows a second diagram of time phenotype analysis in the structure diagram of the multispectral image analysis system, according to the present invention.

FIG. 5 shows a diagram of spectrum analysis for a plurality of target samples in the structure diagram of the multispectral image analysis system, according to the present invention.

### DETAILED DESCRIPTION OF THE PREFERRED EMBODIMENTS

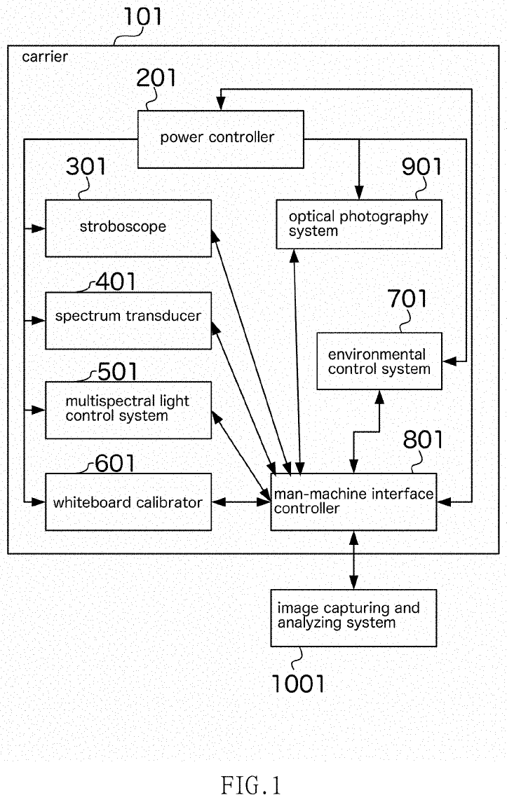

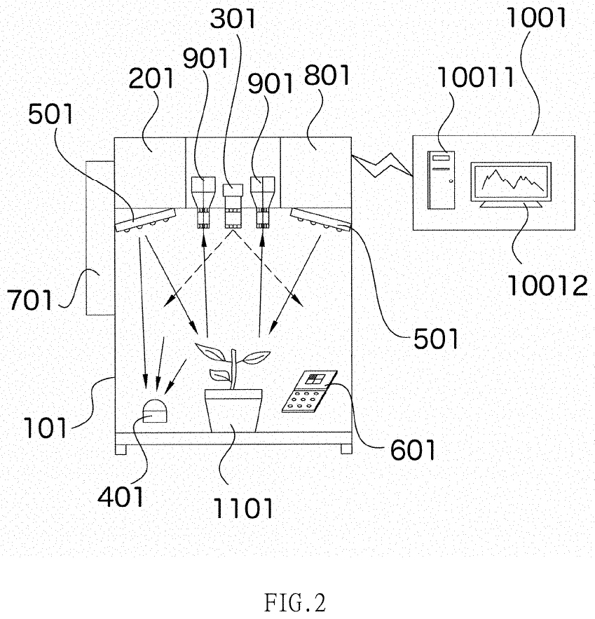

Referring to FIG. 1 first, it shows a structure diagram of a multispectral image analysis system, according to the present invention. In addition, FIG. 2 discloses a structure of the multispectral image analysis system of the present invention. The multispectral image analysis system comprises a carrier 101, which provides a holding space for the placement and cultivation of target samples 1101, and is a containment opened and closed by a cover, accommodating part or all of a power controller 201, a stroboscope 301, a spectrum transducer 401, a multispectral light control system 501, a whiteboard calibrator 601, an environmental control system 701, a man-machine interface controller 801 and an optical photography system 901; the power controller 201, which is connected with the stroboscope 301, the spectrum transducer 401, the multispectral light control system 501, the whiteboard calibrator 601, the environmental control system 701, the man-machine interface controller 801 and the optical photography system 901 to provide the power for operation; the stroboscope 301, which provides a flashlight to irradiate on the target samples 1101, with the reflection light from the target samples 1101 aiding the optical photography system 901 to shoot images; the spectrum transducer 401, which is disposed in an irradiation range of the stroboscope 301 and the multispectral light control system 501 to monitor the wavelength and the illuminance, followed by sending back these data to the man-machine interface controller 801; the multispectral light control system 501, which is disposed above or beside the target samples 1101 to provide a multispectral light source of 16-20 kinds of wavebands in an interval of 25-45 nm, with a power switch being controlled or a light intensity being adjusted by the man-machine interface controller 801; the whiteboard calibrator 601, which is disposed in a shooting range of the lens of the optical photography system 901 to measure the coordinates of the target samples 1101, followed by sending back the measured data to the man-machine interface controller 801 to calibrate the gray level and the focus in height, so as to improve the quality of images to be shot by the optical photography system 901; the environmental control system 701, a part of which is disposed inside and outside the carrier 101 to provide heat exchange between the inner space and the outer space of the carrier 101, providing the inner space of the carrier 101 with a stable environmental condition and monitoring the environmental condition, and sending back the monitoring data to the man-machine interface controller 801; the man-machine interface controller 801, which links bi-directionally with the power controller 201, the stroboscope 301, the spectrum transducer 401, the multispectral light control system 501, the whiteboard calibrator 601, the environmental control system 701 and the optical photography system 901 to control the operation of all parts and receive the real monitoring data of all parts; at least one optical photography system 901, which is disposed above or beside the target samples 1101 to shoot the target samples 1101, followed by sending back the images to the man-machine interface controller 801; and an image capturing and analyzing system 1001, which links bi-directionally with the man-machine interface controller 801 to receive the real monitoring data of all parts from the man-machine interface controller 801 for analysis, followed by sending back the data after analysis to the man-machine interface controller 801 for the compensation or operation of the environmental control parameters. The holding space of the carrier 101 provides for the placement and cultivation of a plurality of target samples 1101 for comparison and analysis. The wavelength of the spectrum of the multispectral light control system 501 is in the range of 330-1100 nm, and the multispectral light control system 501 can adjust and control the switch of a single light source and an intensity of the single light source. The environmental control system 701 includes a temperature controller and a humidity controller to control the environmental condition, such as temperature, humidity or salinity. The man-machine interface controller 801 includes a display unit, a touch control unit and a press-key unit disposed on the outer surface of the carrier, allowing a user to monitor and input the control condition. The optical photography system 901 is capable of shooting 2-D pictures, films, time-lapse photography, 3-D pictures and images for a user to determine, and a plurality of the optical photography system 901 is provided, using hardware with dual lenses to shoot a difference in the depth of field of the target samples 1101 for making a 3-D image at the back end. Furthermore, the optical photography system 901 includes a mechanical arm or a slide rail, allowing the lens of the optical photography system 901 to move the target samples 1101 to be shot, wherein the mechanical arm or the slide rail moves front and back, left and right, or up and down, and provides for setting up and moving the lens and the stroboscope 301. The image capturing and analyzing system 1001 includes a computer host 10011 to compute and control the system, and a screen 10012 which provides for the user to determine the image and access the data. The features of plants analyzed by the image capturing and analyzing system 1001 include the features of spectrum, the growth rate, the pathological analysis, the observation of cultivation phenotype, the diffusion rate, the diffusion and covering area, and the proportion and distribution of phenotype for a plurality of target samples 1101, with the collected images, coordinates and spectrum features providing multi-dimensional data for further data analysis.

The solid arrows in FIG. 2 represent the direction of light transmission, meaning that when the multispectral light control system 501 generates a light source in a specific wavelength to irradiate on the target samples 1101, the spectral transducer 401 and the whiteboard calibrator 601, the target samples 1101 will reflect a reflective light in the specific wavelength to transmit to the lens of the optical photography system 901, allowing the optical photography system 901 to capture the spectra image to act as a reference for the backend image capturing and analyzing system 1001 to analyze the image coordinates and the spectra wavelength. The dashed arrows, on the other hand, represent the flash light released from the stroboscope 301 to irradiate on the target samples 1101, aiding the optical photography system 901 to shoot the images, such as an auxiliary light source for the fluorescence spectrum or an auxiliary light source for the strobe in a specific waveband.

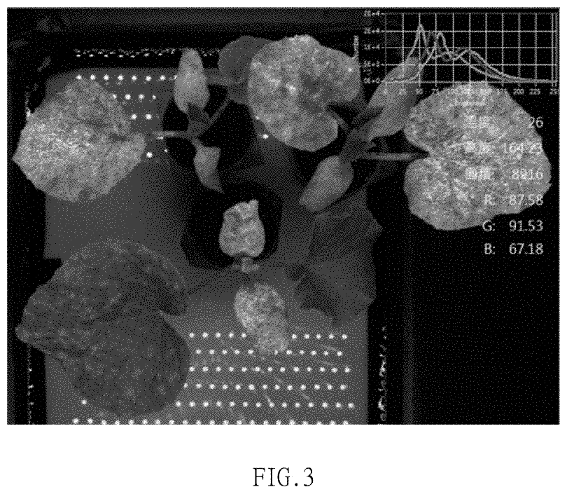

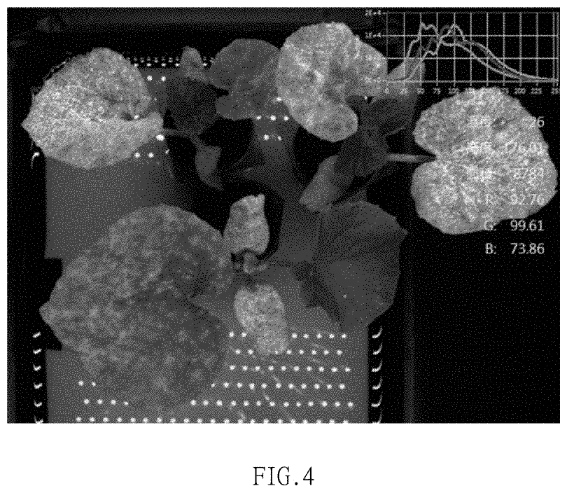

To facilitate further understanding of the scenario in the practical application of the present invention, the analysis and application in the area of plant cultivation are taken as an example. FIG. 3 and FIG. 4 illustrate a first time phenotype analysis diagram and a second time phenotype analysis diagram in the structure diagram of the multispectral image analysis system of the present invention, wherein the plant phenotypes are clearly shot at two different times to determine the cultivation phenotypes under a specific environmental condition. In addition, the waveband information of a specific spectrum on the upper right corner can be used associatively to determine what kind of germ the plant is attacked by during the cultivation. As shown in the drawings, the white dots on the leaves are the areas attacked by the germ; whereas, by these areas, the trend of change in the attacked areas can be observed, so that the pathological analysis, the diffusion rate, and the diffusion and covering area of the plant can be collected for the basis of the determination in the next step. In addition, the present invention can also act as a cultivation space for new drugs or new breeds of creatures, and as there is the environmental control function inside the space, these new drugs or new breeds of creatures can grow rapidly, with a large amount of sample data being able to be accessed rapidly and accurately.

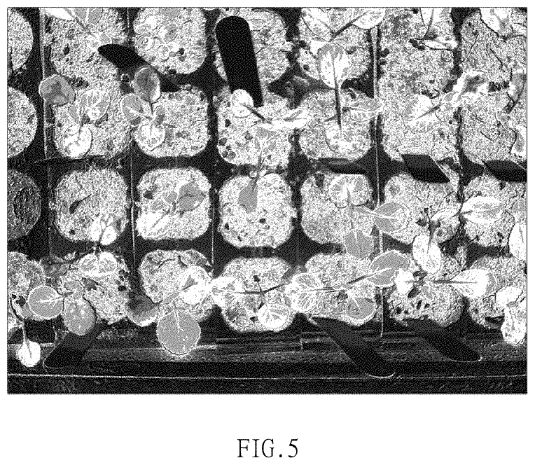

FIG. 5 illustrates a diagram of spectrum analysis for a plurality of target samples in the structure diagram of the multispectral image analysis system according to the present invention, wherein the image analysis can be performed in accordance with the spectral wavebands, so that the physiological or pathological behaviors of a plurality of target samples can be compared in response to the reflection or absorption of the spectra in a certain shooting range. The spectral transducer can detect and send back the wavebands and the illuminance. The images of all kinds of specific germs under a specific multi-spectrum can be entered into a database following a long time of observation, and the images are combined with the spectral data for a large data analysis based upon the multidimensional data, which facilitates a user to observe the growth and damage of the organisms under a variety of conditions. In the present invention, as the plant samples are cultivated in fixed positions for the multispectral analysis, there is no need to move the plant samples and the coordinates of the plant samples will not be changed, which facilitates observing the leaves and the height of the plants at fixed points. In addition, the shooting function can be executed to do time-lapse photography for a single or plurality of plant sample, which enables the user to observe the growth and the change of the plant in the cultivation period in the carrier. Furthermore, for the organisms that are sensitive to light, the organism samples will not be exposed in other spectrum due to the necessity of moving the organism samples, causing the error in the test data. As heat will be generated when the organism samples are irradiated by the spectrum, the present invention is provided with the environmental control system to reduce the error in the environmental condition caused by the radiation heat of the spectrum, so that the organisms can be cultivated and photographed in the carrier for a long time, such as one week, one month or even three months.

The present invention provides the function of cultivating the plural target samples 1101 simultaneously under the environmental control, and provides the light source in a specific waveband using the multispectral light control system 501 to irradiate the target samples. The features of a reflected spectrum in the specific waveband of each coordinate in the image that is shot and marked by the optical photography system are used to determine the physiological and pathological features of each coordinate and to compare the phenotypes and the spectra for the plural target samples, acting as a reference for determining the timing of medication. The environmental control system and the features of multispectral of the present invention are different from that in the prior arts, manifesting the benefits in novelty, advancement and practicality. Therefore, the shortcomings in the prior arts can be improved effectively and there is significant practicality in the application of the present invention.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claimed is:

1. A multispectral image analysis system comprising:
a carrier, which provides a holding space for placement and cultivation of target samples, and is a containment opened and closed by a cover, accommodating part or all of a power controller, a stroboscope, a spectrum transducer, a multispectral light control system, a whiteboard calibrator, an environmental control system, a man-machine interface controller and an optical photography system;
the power controller, which is connected with the stroboscope, the spectrum transducer, the multispectral light control system, the whiteboard calibrator, the environmental control system, the man-machine interface controller and the optical photography system to provide power for operation;
the stroboscope, which provides a flashlight to irradiate on the target samples, with reflection light from the target samples aiding the optical photography system to shoot images;
the spectrum transducer, which is disposed in an irradiation range of the stroboscope and the multispectral light control system to monitor wavelength and illuminance, followed by sending back these data to the man-machine interface controller;
the multispectral light control system, which is disposed above or beside the target samples to provide a multispectral light source of 16-20 kinds of wavebands in an interval of 25-45 nm, with a power switch being controlled or a light intensity being adjusted by the man-machine interface controller;
the whiteboard calibrator, which is disposed in a shooting range of lens of the optical photography system to measure the target samples, followed by sending back measured data to the man-machine interface controller to calibrate gray level and focus in height, thereby improving quality of images to be shot by the optical photography system;
the environmental control system, a part of which is disposed inside and outside the carrier to provide heat exchange between an inner space and an outer space of the carrier, providing the inner space of the carrier with a stable environmental condition and monitoring the environmental condition, and sending back monitored data to the man-machine interface controller;
the man-machine interface controller, which links bi-directionally with the power controller, the stroboscope, the spectrum transducer, the multispectral light control system, the whiteboard calibrator, the environmental control system and the optical photography system to control operation of all parts and receive real monitored data of all parts;
the optical photography system, which is disposed above or beside the target samples to shoot the target samples, followed by sending back images to the man-machine interface controller; and
an image capturing and analyzing system, which links bi-directionally with the man-machine interface controller to receive real monitored data of all parts from the man-machine interface controller for analysis, followed by sending back the data after analysis to the man-machine interface controller for compensation or operation of environmental control parameters.

2. The multispectral image analysis system according to claim 1, wherein the holding space of the carrier provides for placement and cultivation of a plurality of target samples for comparison and analysis.

3. The multispectral image analysis system according to claim 1, wherein the wavelength of the spectrum of the multispectral light control system is in the range of 330-1100 nm.

4. The multispectral image analysis system according to claim 1, wherein the multispectral light control system adjusts and controls the switch of a single light source and an intensity of the single light source.

5. The multispectral image analysis system according to claim 1, wherein the environmental control system includes a temperature controller and a humidity controller to control the environmental condition, such as temperature, humidity or salinity.

6. The multispectral image analysis system according to claim 1, wherein the man-machine interface controller includes a display unit, a touch control unit and a press-key unit disposed on the outer surface of the carrier, allowing a user to monitor and input the control condition.

7. The multispectral image analysis system according to claim 1, wherein the optical photography system is capable of shooting 2-D (2-dimensional) pictures, films, time-lapse photography, 3-D (3-dimensional) pictures and images for a user to determine.

8. The multispectral image analysis system according to claim 1, wherein the optical photography system further includes a mechanical arm or a slide rail, allowing the lens of the optical photography system to move the target samples to be shot.

9. The multispectral image analysis system according to claim 8, wherein the mechanical arm or the slide rail moves front and back, left and right, or up and down, and provides for setting up and moving the lens and the stroboscope.

10. The multispectral image analysis system according to claim 1, wherein features of plants analyzed by the image capturing and analyzing system include the features of spectrum, the growth rate, the pathological analysis, the observation of cultivation phenotype, the diffusion rate, the diffusion and covering area, and the proportion and distribution of phenotype for a plurality of target samples, with the collected images, coordinates and spectrum features providing multi-dimensional data for further data analysis.
